(19) United States
(12) Patent Application Publication
(10) Pub. No.: US 2023/0398019 A1
(43) Pub. Date: Dec. 14, 2023

(54) SYSTEMS AND METHODS FOR PREVENTING OR SLOWING THE PROGRESSION OF MYOPIA WITH A SMART OPHTHALMIC DEVICE

(71) Applicant: TWENTY TWENTY THERAPEUTICS LLC, South San Francisco, CA (US)

(72) Inventors: Zidong Li, South San Francisco, CA (US); Christian Gutierrez, South San Francisco, CA (US)

(21) Appl. No.: 18/209,981

(22) Filed: Jun. 14, 2023

Related U.S. Application Data
(60) Provisional application No. 63/351,842, filed on Jun. 14, 2022.

(51) Int. Cl. A61F 9/00; A61B 3/11; A61K 31/46; A61K 31/5513; A61K 31/4409; A61K 31/5386

(52) U.S. Cl. CPC A61F 9/0017; A61B 3/112; A61K 31/46; A61K 31/5513; A61K 31/4409; A61K 31/5386; A61F 2250/0068; A61B 2017/00066

(57) ABSTRACT

An ophthalmic device can be positioned on a surface of an eye and can modulate an amount of light that can be transmitted therethrough in response to environmental factors and/or release of a therapeutic agent into the eye. The ophthalmic device can include an electronically-mediated medium that can modulate an amount of light transmitted in response to a first electrical signal generated by a signal generator. The ophthalmic device also can include at least one drug reservoir that can release a therapeutic agent to the eye in response to a second electrical signal generated by the signal generator. The signal generator can be encapsulated within the ophthalmic device and in communication with a controller that defines parameters of the electrical signals generated by the signal generator.

### CROSS-REFERENCE TO RELATED APPLICATIONS

This application claims the benefit of U.S. Provisional Application Ser. No. 63/351,842, filed 14 Jun. 2022, entitled “SYSTEMS AND METHODS FOR PREVENTING OR SLOWING THE PROGRESSION OF MYOPIA WITH A SMART OPHTHALMIC DEVICE”, the entirety of which is incorporated by reference for all purposes.

### TECHNICAL FIELD

The present disclosure relates to a smart ophthalmic device, and more specifically, to systems and methods that can prevent or slow the progression of myopia with a smart ophthalmic device that modulates an amount of light transmitted into an eye.

### BACKGROUND

Myopia, also known as nearsightedness, is a disorder that affects at least one in three people, often developing during adolescence. Topical delivery of atropine eye drops, one or more times a day for an extended period of time, has shown promising results in slowing the progression of myopia. However, one of the most common side effects of the atropine eye drops is photophobia, precipitated by atropine's mydriatic effect, allowing an uncomfortable amount of light to enter the eye. Photophobia results in significant pain and discomfort, which causes many users to stop using the atropine eye drops entirely. The single most effective tool for treating photophobia is to physically reduce the overall light intensity entering the eye and is traditionally done with sunglasses/other tinted glasses and/or limiting exposure to light areas. However, continuous wear of sunglasses/other tinted glasses and/or staying in darkened areas continuously are not practical solutions to address photophobia symptoms especially for adolescents.

### SUMMARY

Described herein are systems and methods for modulating light transmission through a smart ophthalmic device (e.g., a smart contact lens) to prevent photophobia symptoms. The light can be modulated in response to environmental factors and/or the release of a therapeutic agent for preventing or slowing myopia progression into the eye, as an alternative to always wearing sunglasses/other tinted glasses and/or staying in darkened areas. The systems and methods can provide an effective and timely dosage of a therapeutic agent to prevent and/or slow the development of myopia and then modulate the amount of light transmission to prevent the photophobia symptoms.

In one aspect, the present disclosure includes a system that can electronically modulate an amount of light transmitted to a portion of an eye through an ophthalmic device. The ophthalmic device, positionable on a surface of the eye, comprises an electronically-mediated medium that is configured to modulate an amount of light transmitted through a portion of the ophthalmic device in response to a first electrical signal from a signal generator. The signal generator is encapsulated within the ophthalmic device and configured to generate the first electrical signal. The signal generator is in communication with a controller, which comprises a processor, that is configured to define parameters of the first electrical signal. It should be noted that the amount of light transmitted through the ophthalmic device can be regulated in concert with a drug being released from the ophthalmic device.

In another aspect, the present disclosure includes a method for programmable therapeutic agent delivery and programmable light modulation for preventing or slowing down the progression of myopia. An ophthalmic device having at least one encapsulated drug reservoir configured to store a therapeutic agent covered by a metal electrode, and comprising an electronically-mediated medium, is positioned on a surface of an eye. A first electrical signal is delivered to the metal electrode covering a certain drug reservoir of the at least one encapsulated drug reservoir so the metal electrode undergoes electrodissolution to release the therapeutic agent. The therapeutic agent is configured to cause dilation of a pupil of the eye. After a pupil dilation time has elapsed the electronically-mediated medium is activated to reduce the amount of light transmitted through a portion of the ophthalmic device by filtering the light. The electronically-mediated medium activates in response to receiving a first portion of a second electrical signal. After a pupil recovery time has elapsed, the amount of light transmitted through the portion of the ophthalmic device is returned to normal by deactivating the electronically-mediated medium. The electronically-mediated medium deactivates in response to receiving a second portion of the second electrical signal, which has a zero voltage or a voltage of an opposite polarity from the first portion of the second electrical signal.

In another aspect, the present disclosure includes another method for programmable therapeutic agent delivery and programmable light modulation for preventing or slowing the progression of myopia. An ophthalmic device having at least one encapsulated drug reservoir configured to store a therapeutic agent covered by a metal electrode, and comprising an electronically-mediated medium, is positioned on a surface of an eye. A first electrical signal is delivered to the metal electrode covering a certain drug reservoir of the at least one encapsulated drug reservoir so the metal electrode undergoes electrodissolution to release the therapeutic agent. The therapeutic agent is configured to cause dilation of a pupil of the eye. When a pupil dilation time has elapsed, ambient light near the eye at a given time is detected and a first portion of a second electrical signal is delivered to the electronically-mediated medium in response to the detected ambient light at the given time. The first portion of the second electrical signal can comprise a variable waveform. The electronically-mediated medium undergoes a change to modulate an amount of light transmitted through at least a portion of the ophthalmic device. When a pupil recovery time has elapsed a second portion of the second electrical signal is delivered to the electronically-mediated medium to return the electronically-mediated medium to transmitting a normal amount of the light through the portion of the ophthalmic device.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a diagram of system that can modulate an amount of light entering an eye of a user through a smart ophthalmic device;

FIG. 2 shows a diagram of a system that can deliver a therapeutic agent to an eye of a user and modulate an amount of light entering the eye through a smart ophthalmic device;

FIG. 3 shows examples of two different types of electronically-mediated medium materials that can be used in the systems of FIGS. 1 and 2 and how the materials can function;

FIG. 4 shows a pictographic example of release of a therapeutic agent from a drug reservoir of a smart ophthalmic device of the system of FIG. 2;

FIGS. 5-7 show examples of the instructions and control logic of the system of FIG. 1 or 2 executable by the controller to activate the accompanying light modulation of the smart ophthalmic device;

FIG. 8 shows example waveforms that can be applied via the system of FIG. 1 or 2;

FIG. 9 shows an example embodiment of the system shown in FIGS. 1 and 2;

FIG. 10 shows an example process flow diagram of a method of modulating an amount of light transmitted through a portion of a smart ophthalmic device;

FIG. 11 shows an example process flow diagram of a method of delivering a therapeutic agent to an eye of a user and modulating an amount of light entering the eye through a smart ophthalmic device;

FIGS. 12 and 13 show example process flow diagrams of methods of delivering a therapeutic agent to an eye of a user and modulating an amount of light entering the eye.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ophthalmic device” refers to a medical instrument used on or within a portion of a patient's eye for optometry or ophthalmology purposes (e.g., for diagnosis, surgery, vision correction, or the like). An ophthalmic device can be “smart” when it includes one or more components that facilitate one or more active processes for purposes other than traditional lens-based vision correction (e.g., therapeutic agent release or modulation of light transmission). Unless otherwise stated, as used herein, the term “ophthalmic device” should be understood to mean “smart ophthalmic device”. As an example, the “smart ophthalmic device” can be a smart contact lens. Smart can refer to a device with at least one processing capability and/or electronically connected component.

As used herein, the term “reservoir” refers to a storehouse for a therapeutic that includes a well being open for release of the therapeutic agent (allowing for diffusion of the therapeutic agent out of the reservoir and eventually into an eye of a person wearing the ophthalmic device). The opening of the well may be covered to prevent release of the therapeutic agent. In some instances, the covering can be a metal electrode that facilitate release of the therapeutic agent from the reservoir. For example, the metal electrode can electrodissolve in response to an electrical signal to facilitate the release of the therapeutic agent.

As used herein, the term “electrical signal” refers to a signal waveform generated by an electronic means, such as a signal generator. An electrical signal may be a voltage signal or a current signal. An electrical signal can have variable parameters including, but not limited to, frequency, magnitude, shape, amplitude, and polarity. The variable parameters can be controlled, for example, by a controller in communication with the signal generator.

As used herein, the term “electronically-mediated medium” refers to any material that undergoes a change in light transmissibility in response to an electrical signal. Non-limiting examples of electrical-mediated materials include: electrochromic materials, liquid crystal materials, pH sensitive materials, or thermodynamic materials.

As used herein, the term “therapeutic agent” refers to one or more substance (e.g., liquid, solid, or gas) related to the prevention, treatment, symptom relief, and/or palliative care of a disease, injury, or other malady. The therapeutic agent can be a pharmaceutical, for example. The therapeutic agent can be stored, for example, in a non-liquid form, such as a powder or gel. For slowing the progression of and/or preventing myopia the therapeutic agent can be, for example, a muscarinic receptor antagonist, such as, but not limited to, atropine, pirenzepine, tropicamide, or scopolamine.

As used herein, the terms “subject”, “user”, and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

As used herein, the term “ambient light” refers to the available light in an environment (e.g., in the environment near a subject). The illuminance of ambient light can be detected, for example, with an ambient light sensor, such as, but not limited to, a phototransistor, a photodiode, or a photonic integrated circuit. Illuminance is the measure of the quantity of incident light that illuminates a surface and refers to the total luminous flux incident on the surface per unit area (SI Unit=lux, Dimension=L⁻² J).

### II. Overview

Topical delivery of pharmaceuticals, such as atropine eye drops, particularly at a high concentration have shown promising results in slowing, or even preventing, the progression of myopia. Photophobia, a common side effect of therapeutic agents, such as pharmaceuticals like atropine, can cause pain and discomfort, leading to a low compliance rate and high dropout rate during clinical studies. The single-most effective tool for treating photophobia is to physically reduce the overall light intensity entering the eye, such as using sunglasses and/or photochromic tinted lenses and/or staying in a darker room. However, this is inconvenient and leads to a low compliance, especially for children. Therefore, integrating high concentration pharmaceutical delivery and photophobia side effect reduction is a significant key to enabling effective slowing and/or prevention of myopia.

Systems and methods are described that enable programmable delivery of a therapeutic agent, such as atropine, for effective myopia treatment from a smart ophthalmic device while modulating light transmission through the smart ophthalmic device, via an electronically-mediated medium. The systems and methods can operate to prevent excessive light entering the eye to reduce photophobia symptoms that can be caused by the therapeutic agent. The systems and methods can control delivery of the therapeutic agent from one or more drug reservoirs (e.g., enough reservoirs for delivery of the therapeutic agent for a day, a week, a month, etc.) of the ophthalmic device, thus removing the need for eye drops and daily patient compliance. Delivering a therapeutic agent via a smart ophthalmic device also increases the residence time of the therapeutic agent on the eye, which increases the efficacy of the therapeutic agent as compared to eye drops. The systems and methods can control the transmission of light through the electronically-mediated medium of the smart ophthalmic device based on timing of pupil dilation after the therapeutic agent is released and/or the amount of ambient light within a user's environment at a given time, thus reducing or eliminating the effects of photophobia.

### III. Systems

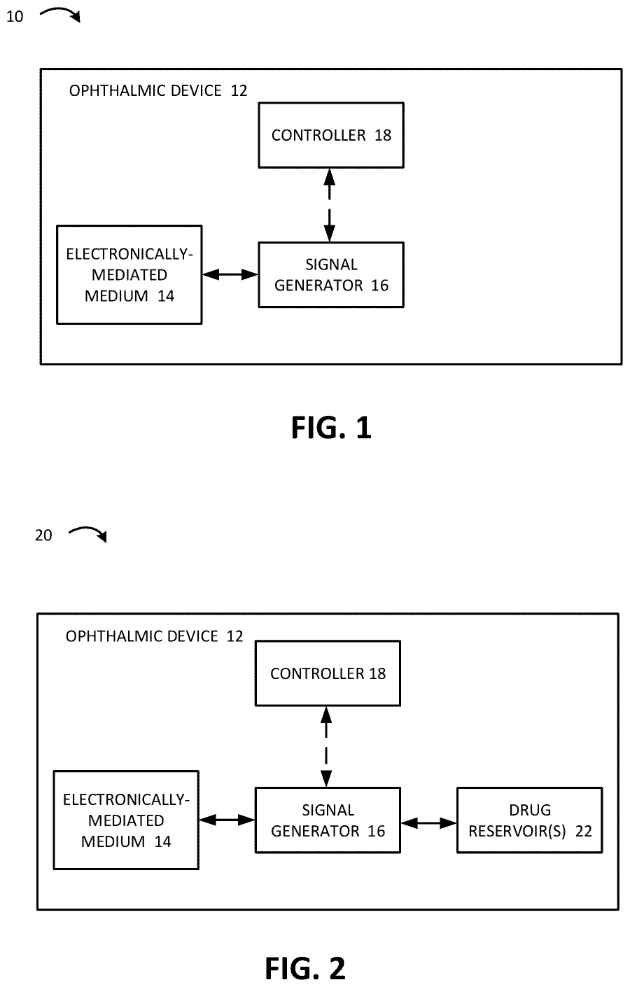

Provided herein are systems 10, 20 (FIGS. 1, 2) that can modulate an amount of light transmitted through a smart ophthalmic device to prevent photophobia symptoms. As shown in FIG. 1, the system 10 can modulate an amount of light in response to environmental factors, with or without the application of a therapeutic agent. The system 20 (FIG. 2) can also prevent or slow the progression of myopia. As shown in FIG. 2, the system 20 can provide effective and timely dosage of a therapeutic agent for the prevention and/or slowing of myopia while modulating an amount of light transmitted through the contact lens to prevent photophobia symptoms.

The system 10, shown in FIG. 1, can include an ophthalmic device 12 that can be positioned on an eye of a subject (e.g., a smart contact lens). The ophthalmic device 12 can have body made of a hydrogel matrix with components (e.g., electronically-mediated medium 14, signal generator 16, etc.) embedded and/or encompassed therein. The hydrogel matrix can be formed of a hydrogel-based material and water. The hydrogel-based material can be any cross-linked hydrophilic polymer that does not dissolve in water. Accordingly, the hydrogel-based material can be stiff when dry, but soft and pliable when hydrated. The hydrogel-based material can be highly absorbent, and can have a naturally-high water content (e.g., 20%-60%), yet maintain a well-defined structure. Non-limiting examples of hydrogel-based material monomers are hydroxyethylmethacrylate (HEMA) or derivatives, methacrylic acid (MA) or derivatives, methyl methacrylate (MMA) or derivatives, n-vinyl pyrrolidone (NVP) or derivatives, poly vinyl alcohol (PVA) or derivatives, polyvinyl pyrrolidone (PVP) or derivatives, and the like. In some instances, the hydrogel-based material can include silicone (as a “silicone-hydrogel”), increasing the oxygen transmissibility and permeability of the hydrogel (among other bulk and surface properties that the presence of silicone improves).

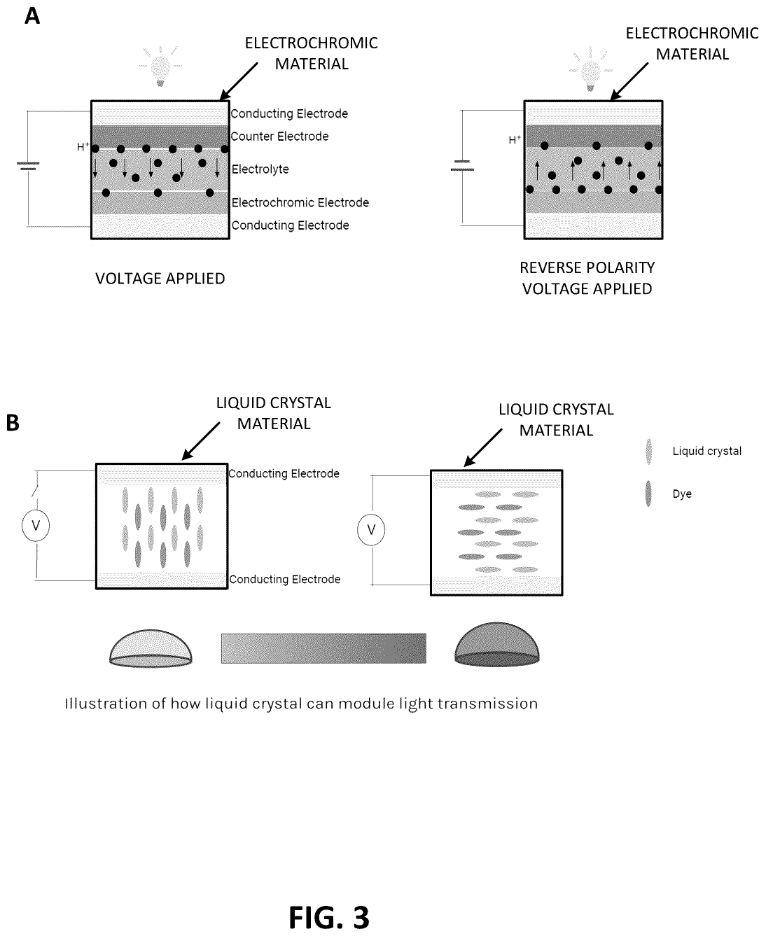

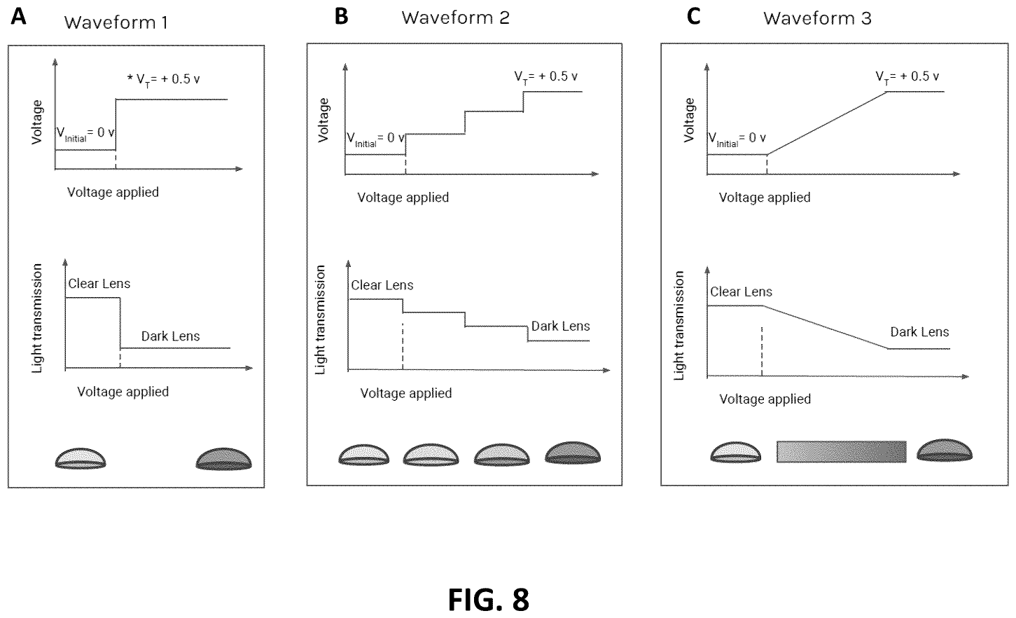

The ophthalmic device 12 can include an electronically-mediated medium 14 and a signal generator 16. The electronically-mediated medium 14 can be configured to modulate an amount of light transmitted through a portion of the ophthalmic device in response to a first electrical signal from the signal generator 16 (non-limiting examples of this modulation are shown in FIG. 3). The first electrical signal can have an alternative current or a direct current, and the first electrical signal may have a variable waveform. Exemplary electrical signal waveforms are shown in FIG. 8. The signal generator 16 can transmit the first electrical signal over a wired connection, a wireless connection, or a combination of wired and wireless connection to, for example, the electronically-mediated medium 14, a component associated with the electronically-mediated medium 14, or the like). The electronically-mediated medium 14 can be, for example, a material that is one of electrochromic, liquid crystal, pH sensitive, or thermodynamic. The electronically mediated medium 14 can be included in the ophthalmic device 12 in any manner suitable to modulate the amount of light that can transmit through a portion of the ophthalmic device to at least a pupil of an eye the ophthalmic device is positioned on. The signal generator 16 can be encapsulated within the ophthalmic device (e.g., within a hydrogel-based body of the ophthalmic device) and is configured to generate electrical signals, such as the first electrical signal. The signal generator 16 can be in communication (wired or wireless) with a controller 18, which can include a processor and, optionally, a memory (not shown).

The controller 18 can define the parameters (e.g., frequency, magnitude, amplitude, shape, polarity, and the like) of the electrical signals generated by the signal generator 16, such as the first electrical signal. The signal generator 16 can send the first electrical signal to the electronically-mediated medium 14 to modulate the amount of light transmitted through the portion of the ophthalmic device 12 in response to an automatic command (e.g., based on a time and/or environment based control loop programmed in the controller 18) or a manual command (e.g., based on a user generated command to the controller, such as via a mobile device in communication with the controller or directly input into the controller). The controller 18 can be encapsulated within the ophthalmic device 12 (as shown), external from the ophthalmic device (not shown), or embodied at least partially within the ophthalmic device (not shown).

The controller 18 can store instructions (e.g., computer executable instructions) related to defining and controlling the application of electrical signals via the signal generator in a memory (not shown). The controller 18 can also store additional instructions, data, and information. For example, the controller 18 can be an application-specific integrated circuit (ASIC) or other device that includes control circuitry that resides within the ophthalmic device 12. The controller 18 can include at least a system bus, a communication link, a processor (or processing unit), and a memory, that can be one or more non-transitory memory devices. The processor can be, for example, embedded within one or more application specific integrated circuits (ASICs), digital signal processors (DSPs), digital signal processing devices (DSPDs), programmable logic devices (PLDs), field programmable gate arrays (FPGAs), microprocessors, other electronic units designed to perform the functions of a processor, or the like. However, in some instances the memory and the processor can be embodied within the same device (e.g., a microcontroller device).

As shown in FIG. 2, the system 20 can include at least one drug reservoir (drug reservoir(s) 22) encapsulated within the ophthalmic device 12 in addition to the electronically-mediated medium 14, the signal generator 16, and the controller 18. The signal generator 16 can send the first electrical signal to the electronically-mediated medium 14 (or to something associated with the electronically-mediated medium) to modulate the amount of light transmitted through the portion of the ophthalmic device 12, as described with respect to FIG. 1.

The at least one drug reservoir 22 can store a dose of a therapeutic agent and can release the therapeutic agent in response to receiving a second electrical signal from the signal generator 16. The signal generator 16 can generate the second electrical signal to release the therapeutic agent. The second electrical signal can have an alternating current or a direct current, and may have a variable waveform. The signal generator 16 may generate the second electrical signal in response to a programmed time command of the controller 18 (e.g., time for a daily dose of the therapeutic agent, so generate a second electrical signal and apply it to one or more drug reservoirs) and send the second electrical signal to the at least one drug reservoir 22. The controller 18 can define the parameters (e.g., frequency, magnitude, amplitude, shape, polarity, and the like) of the second electrical signal. The therapeutic agent stored in the at least one drug reservoir 22 can be a muscarinic receptor antagonist for preventing or slowing the progression of myopia. For example, the therapeutic agent can be one or more of atropine, pirenzepine, tropicamide, or scopolamine.

For example, the therapeutic agent can be atropine (e.g., with a concentration of 1% or less, 2% or less, or the like). Topical delivery of atropine eye drops has shown promising results in slowing, or even preventing, the progression of myopia. Higher concentrations of atropine in eye drops are reported to be more effective in slowing down the progression of myopia. During a 2-year study of 400 children the mean myopia progression was −0.30, −0.38, and −0.49 D in the atropine 0.1%, and 0.01% groups, respectively. While in another study by the same group, average myopia progression among the 400 children was −0.28 D in atropine 1% group. Other studies have reported similar findings. For example, another group reported the atropine was the most effective among 0.5%, 0.25% and 0.1% groups. During the 2-year study, 61% of the children in the 0.5% atropine group had no progression of myopia while for this rate dropped to 49% and 42% for lower concentration 0.25% and respectively.

The most frequently reported side effects for atropine eye drops are photophobia, blurred vision, local allergic response, and headache. Photophobia can cause pain and discomfort because too much light can enter the pupil. In a study conducted in Asia, 100% of the 247 children who received 1% atropine eye drops reported photophobia and more than 50% reported photophobia as the main reason for dropping out of the study. In another study conducted in Europe with a 0.5% atropine eye drop, photophobia was also the most prominently reported adverse event with 72% of the children in the study reporting experiencing photophobia.

Higher concentrations of atropine eye drops are more effective at slowing the progression or myopia, but also dilate the pupil more. In one study, the mean mesopic pupil sizes increased from 4.7 mm baseline (no atropine) to 5.5 mm, 6.9 mm, and 7.8 mm for 0.01%, 0.1% and 0.5% atropine drops respectively. Larger dilated pupils pass more light through, therefore causing more severe photophobia. In one study, 100% of the patients reported photophobia using 1% atropine eye drops, while in another study 72% of the patients reported photophobia using 0.5% atropine eye drops. As the concentration of atropine lowers, the rate of photophobia continues to drop. A third study reported only 7% of the patients having complaints about photophobia using atropine eye drops and 0% using 0.1% atropine eye drops. Methods and systems for delivering effective concentrations of atropine and for limiting the effects of photophobia symptoms, as described herein, are therefore necessary for safe and effective delivery of atropine for slowing and/or preventing the progression of myopia.

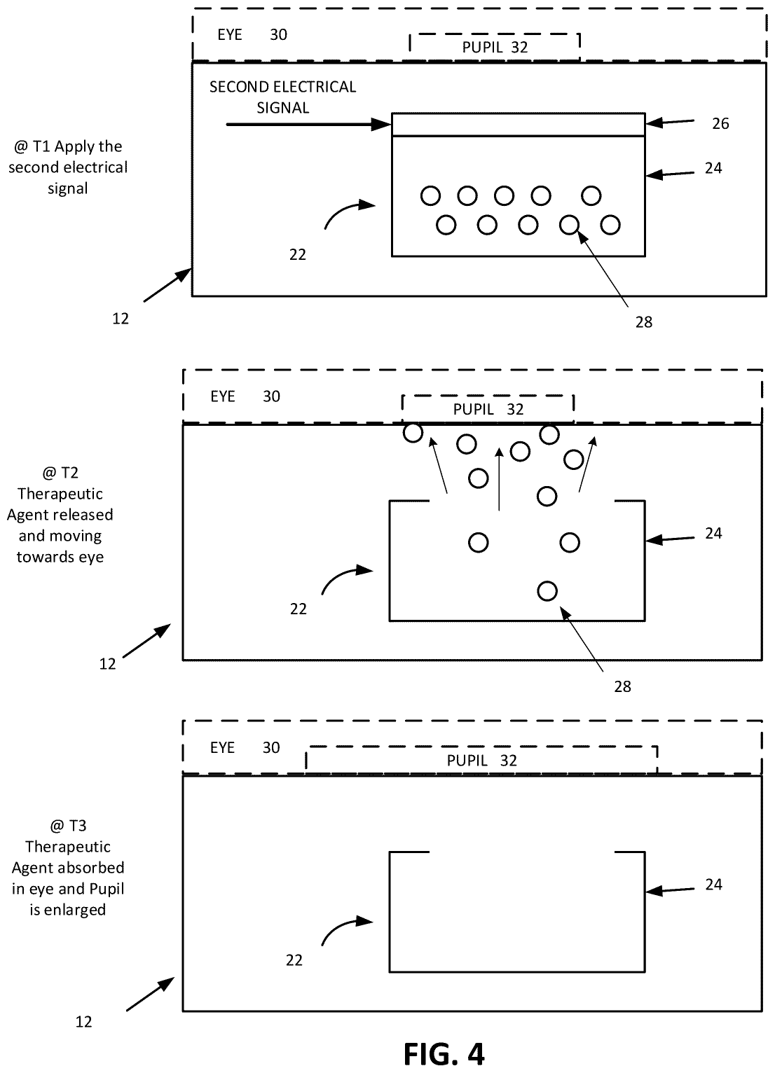

FIGS. 3 and 4 show examples of the electronically-mediated medium 14 (of FIGS. 1 and 2) and the drug reservoir(s) (of FIG. 2) that can modulate light transmission into the eye and deliver the therapeutic agent, such as atropine, in more detail. FIG. 3 shows pictographic examples of electronically-mediated medium material and how the electronically-mediated medium material can be activated or deactivated according to the electrical signal applied. The electrical signal can be applied to one or more electrodes (e.g., conducting electrodes) that make up the material of the electronically-mediated medium. The electronically mediated medium can be a material that is one of electrochromic, liquid crystal, pH sensitive, or thermodynamic. Examples of pH sensitive material include pH responsive co-polymers that can change light transmission, for example, P4VP-b-PMAA (poly(4-vinylpyridine)-block-poly(methacrylic acid)), that can be made either by immobilizing a pH-sensitive organic dye (FITC-dextran, Pyranine (HPTS) etc.) or pH-responsive linkers (poly(acrylic acid) (PAA); poly(2-vinylpyridine) (P2VP)) in polymer. For example, poly(2-vinylpyridine) (P2VP), polyaniline, Pyranine (HPTS). Examples of thermodynamic materials include thermochromatic liquid crystals, thermochromic leuco dyes, thermochromic polymers with embedded thermochromic additives, or the like.

FIG. 3, element A shows an example with an electrochromic material. The electrochromic material includes a conducting electrode on the outer side of a counter electrode on one side (e.g., the side facing away from the eye) and an electrochromic electrode on an inside of a conducting electrode on the other side (e.g., the side facing the eye) with an electrolyte sandwiched between the counter electrode and the electrochromic electrode. When a voltage is of a give polarity is applied to the conducting electrode(s) the H+ ions in the electrolyte flow in one direction, allowing light to transmit normally though the electrochromic material. When a voltage of an opposite polarity is applied the H+ ions in the electrolyte flow in an opposite direction, reducing the amount of light that can be transmitted through the electrochromic material.

FIG. 3, element B shows an example of a liquid crystal material, which includes liquid crystals and a dye sandwiched between two conducting electrodes. When no voltage is applied to the conducting electrodes, then light transmits normally through the liquid crystal material (liquid crystals and dye). When a voltage is applied, and in some instances dependent on the strength or polarity of the voltage applied, the direction of the liquid crystal changes, forcing the direction of the dye to change and the amount of light transmitted through the liquid crystal material is reduced. Besides reducing light intensity through a portion of an ophthalmic device, certain color modulation may be added to enhance relieving photophobia, for example, a pink-colored lens filters green and blue lights which has been shows to relieve the pain of photophobia.

FIG. 4 shows an example of how one drug reservoir 22 (of FIG. 2) can store and then release a therapeutic agent, such as atropine or another muscarinic receptor antagonist, for delivery onto the surface and/or into a portion of the eye. As shown in the block diagram of FIG. 4, each of drug reservoir(s) 22 encapsulated within the ophthalmic device 12 can include a well 24 having an interior configured to hold the therapeutic agent 28. The therapeutic agent 28 can be in a non-liquid form (e.g., a powder, a gel, or the like). A metal electrode 26 can cover an opening of the well 24 and can electrodissolve in response to receiving the second electrical signal from the signal generator (not shown in FIG. 4). The metal electrode 26 can include one or more electrochemically active metals, for example, the metal electrode can be a gold film electrode.

At time T1 the second electrical signal, generated by the signal generator, can be applied to the metal electrode 26 of the at least one drug reservoir 22 to activate electrodissolution of the metal electrode so as to release the therapeutic agent 28. At time T2 the metal electrode 26 is dissolved and the therapeutic agent 28 is released to contact or enter the eye 30, including pupil 32, through any suitable manner of movement, such as diffusion. At times T1 and T2 the pupil 32 is not affected by the therapeutic agent 28 (e.g., not dilated because of the therapeutic agent). At time T3 the therapeutic agent 28 has been absorbed by the eye 30 and pupil 32 is dilated (e.g., enlarged) as an effect of the therapeutic agent.

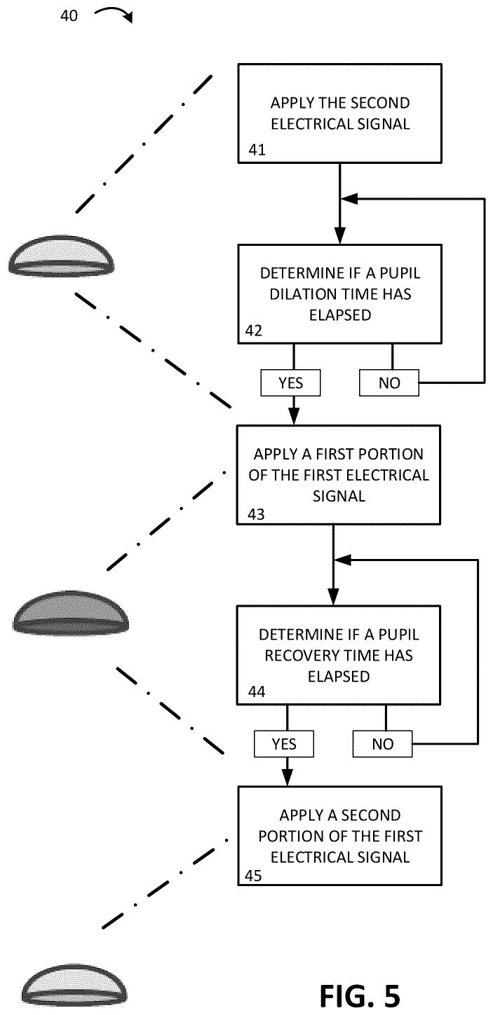

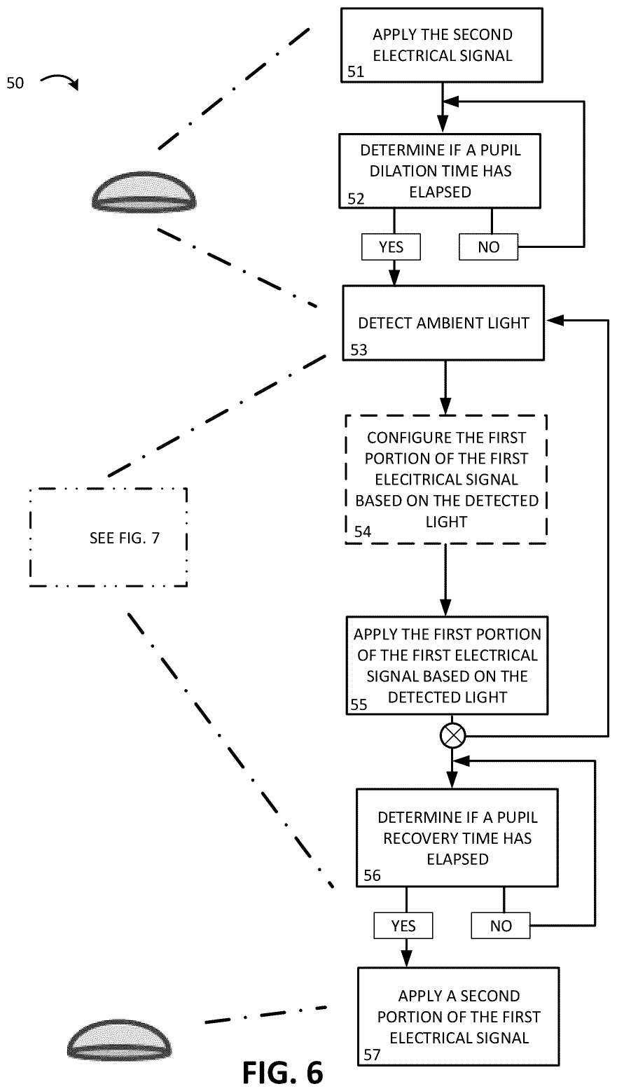

FIGS. 5 and 6 show example flow diagrams 40 and 50 of the logic and instructions the controller 18 can execute in order for the system 20 to deliver a therapeutic agent 28 to prevent and/or slow the progress of myopia while reducing the negative effects experienced by a user of the system 10 because of the therapeutic agent. FIG. 5 shows an example of a time-based control loop and FIG. 6 shows an example of an environment-based control loop. FIGS. 5 and 6 both also indicate the state of the electronically-mediated medium 14 (e.g., pictographs of activated (dark) or not activated (clear) ophthalmic devices) at the different time points when the instructions are executed.

Referring to FIG. 5, at 41, the controller 18 executes the instructions to the signal generator 16 to generate and apply the second electrical signal to the at least one drug reservoir 22 to activate electrodissolution of the metal electrode 26 (as shown in FIG. 4) so as to release the therapeutic agent 28. At 42, the controller 18 can execute the instructions to determine if a pupil dilation time has elapsed since release of the therapeutic agent 28 (that causes pupil dilation) was triggered by application of the second electrical signal (e.g., based on known electrodissolution speeds and known diffusion and activation speeds of the therapeutic agent). If the pupil dilation time has not yet elapsed then the controller 18 can check again at a later time (e.g., 1 second, 30 second, 1 minute, 5 minutes etc.) to see if the pupil dilation time has elapsed yet. When the pupil dilation time has elapsed, the controller 18 can execute the instructions, at 43, for the signal generator 16 to generate and apply a first portion of the first electrical signal to the electronically-mediated medium 14 to modulate the amount of the light transmitted through the portion of the ophthalmic device 12. After application of the first portion of the first electrical signal for a time to the electronically-mediated medium 14 then the electronically-mediated medium is activated to reduce the amount of light transmitted through the portion of the ophthalmic device 12 (e.g., the ophthalmic device can darken, as shown).

The electronically-mediated medium 14 can remain activated until the controller 18 can determine, at 44, if a pupil recovery time has elapsed (e.g., based on known times it takes for the dilation effects of the therapeutic agent 28 to wear off). After the controller 18 determines the pupil recovery time has elapsed, then the controller can instruct the signal generator 16, at 45, to generate and apply a second portion of the first electrical signal to the electronically-mediated medium 14 to return the electronically-mediated medium to transmitting a normal amount of the light through the portion of the ophthalmic device 12. The first part of the first electrical signal can have a direct current or an alternating current. The second part of the first electrical signal can have zero voltage if the first part of the first electrical signal has either a direct current or an alternating current. The second part of the first electrical signal can, in some instances, have a reverse (or inverse) polarity from the first part of the first electrical signal if the first part of the first electrical signal has a direct current. The second electrical signal can also be a direct current or an alternating current.

Referring now to FIG. 6, which shows an environment-based control loop executed by controller 18, the systems 10 and 20, shown in FIGS. 1 and 2, can further include an ambient light sensor. The ambient light sensor can be encapsulated in ophthalmic device 12 or can be external to ophthalmic device (e.g., on glasses worn by the user or otherwise attached to a user's person or carried by a user). At 51, the controller 18 executes the instructions to the signal generator 16 to generate and apply the second electrical signal to the at least one drug reservoir 22 to activate electrodissolution of the metal electrode 26 (as shown in FIG. 3) so as to release the therapeutic agent 28. At 52, the controller 18 can execute the instructions to determine if a pupil dilation time has elapsed since release of the therapeutic agent 28 (that causes pupil dilation) was triggered by application of the second electrical signal (e.g., based on known electrodissolution speeds and known diffusion and activation speeds of the therapeutic agent). If the pupil dilation time has not yet elapsed then the controller 18 can check again at a later time (e.g., 1 second, 30 second, 1 minute, 5 minutes etc.) to see if the pupil dilation time has elapsed yet. After the pupil dilation time has elapsed, the controller 18 can execute instructions, at 53, to detect ambient light via the ambient light sensor at a given time. The ambient light sensor can continuously detect ambient light or can detect ambient light after a time period has passed (e.g., 1 second, 10 seconds, 30 second, 1 minute, 5, minutes, etc.).

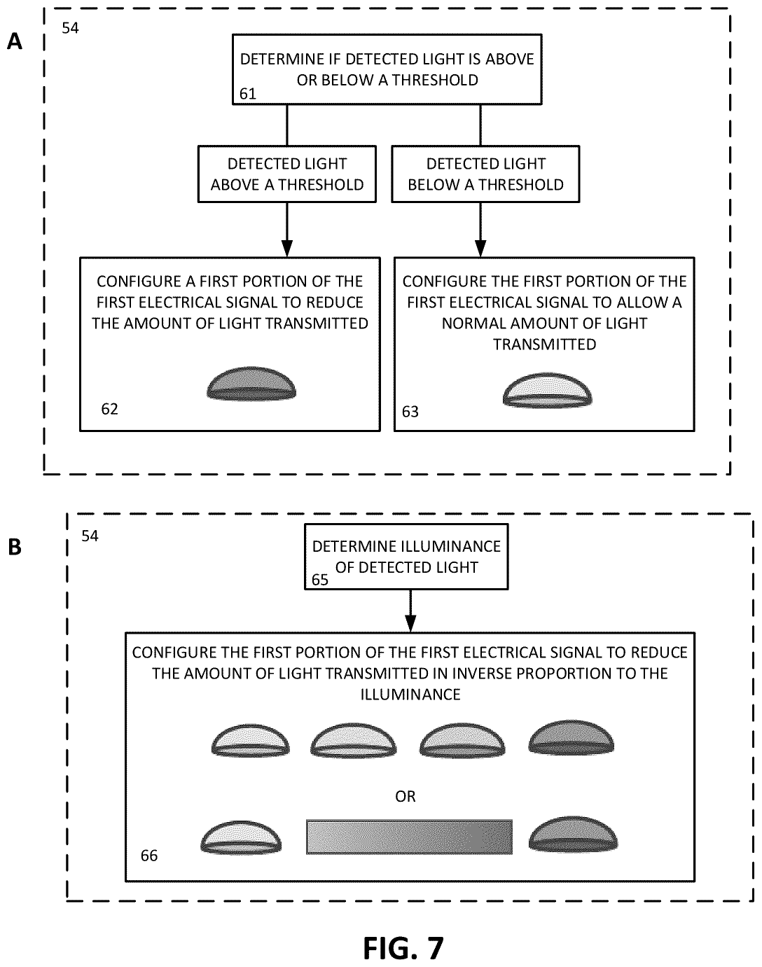

At 54, the controller 18 can configure the first portion of the first electrical signal based on the detected ambient light to modulate the amount of light transmitted through the portion of the ophthalmic device in response to the detected ambient light. Shown in more detail in FIG. 7. At 55, the controller 18 can execute the instructions for the signal generator 16 to generate and apply the first portion of the first electrical signal to the electronically-mediated medium 14 to modulate the amount of the light transmitted through the portion of the ophthalmic device 12. The controller 18 can continue to detect and reconfigure the first portion of the first electrical signal for a given time (e.g., in increments of 30 seconds, 1 minute, 5 minutes, etc.) until the controller has determined the pupil recovery time has elapsed, at 56. After the controller 18 determines the pupil recovery time has elapsed, then the controller can instruct the signal generator, at 57, to generate and apply a second portion of the first electrical signal to the electronically-mediated medium 14 to return the electronically-mediated medium to transmitting a normal amount of the light through the portion of the ophthalmic device 12. The control loop of FIG. 6 can be used, for example, when a user is moving between environments with significantly different ambient light amounts and always filtering light would be undesirable, such as if a user is in class (darker light environment) then goes to soccer practice (brighter light environment) and then to a movie theater (darker light environment) while the therapeutic drug is causing pupil dilation.

FIG. 7 shows two methods for configuring the first portion of the first electrical signal according to the instructions 54 executed by controller 18 in control loop 50. FIG. 7, element A shows a thresholding approach to determining if the electronically-mediated medium should be activated or not based on the detected ambient light. The threshold can be based on known data for what amount of light causes adverse photophobic effects. The controller 18 can execute instructions, 61, to determine whether the detected ambient light is above or below a threshold value at a given time. If the detected ambient light is above the threshold, then the controller 18 can execute instructions to configure the first portion of the first electrical signal to reduce the amount of light transmitted through the portion of the ophthalmic device 12 by the electronically-mediated medium 14 (e.g., have a positive voltage or an alternating current with a given frequency and/or magnitude). If the detected ambient light is below the threshold, then the controller 18 can execute instructions, 63, to configure the first portion of the first electrical signal to return, or keep, the amount of light transmitted through the portion of the ophthalmic device 12 by the electronically-mediated medium 14 to normal (e.g., have a zero voltage or a reverse polarity compared to the positive voltage).

FIG. 7, element B shows a stepped or graduated approach to determining if the electronically mediated medium 14 should be activated or not based on the detected ambient light. The controller 18 can execute instructions, 65, to determine the illuminance of the detected ambient light at the given time. The controller 18 can then execute instructions, 66, to configure the first portion of the first electrical signal to reduce, or increase, the amount of light in inverse proportion to the illuminance. For example, the first portion of the first electrical signal can be a step function, where the electronically-mediated medium 14 activates in various distinct grades of filtration for given ranges of illuminance. In another example, the first portion of the first electrical signal is a ramp function, wherein the electronically-mediated medium 14 activates in a graded fashion for each quantity of illuminance detected.

Examples of different voltage waveforms that can be generated and applied by the signal generator are shown in FIG. 8. FIG. 8, element A shows a basic one-step voltage waveform that can either activate or deactivate the electronically-mediated medium 14, which can be used in either the time or the environment-based control loops. When the voltage is applied, the light transmission is reduced. If the voltage is stopped or a reverse voltage is applied, then the light transmission would revert to normal. FIG. 8, element B shows a multi-step step function that can partially activate or deactivate electronically-mediated medium to alter the light transmission in several filter options based on different voltages applied (e.g., different voltages for different ranges of detected illuminance). FIG. 8, element C shows a ramp function that can partially activate or deactivate the electronically-mediated medium to alter the light transmission in a gradient approach based on the illuminance values detected.

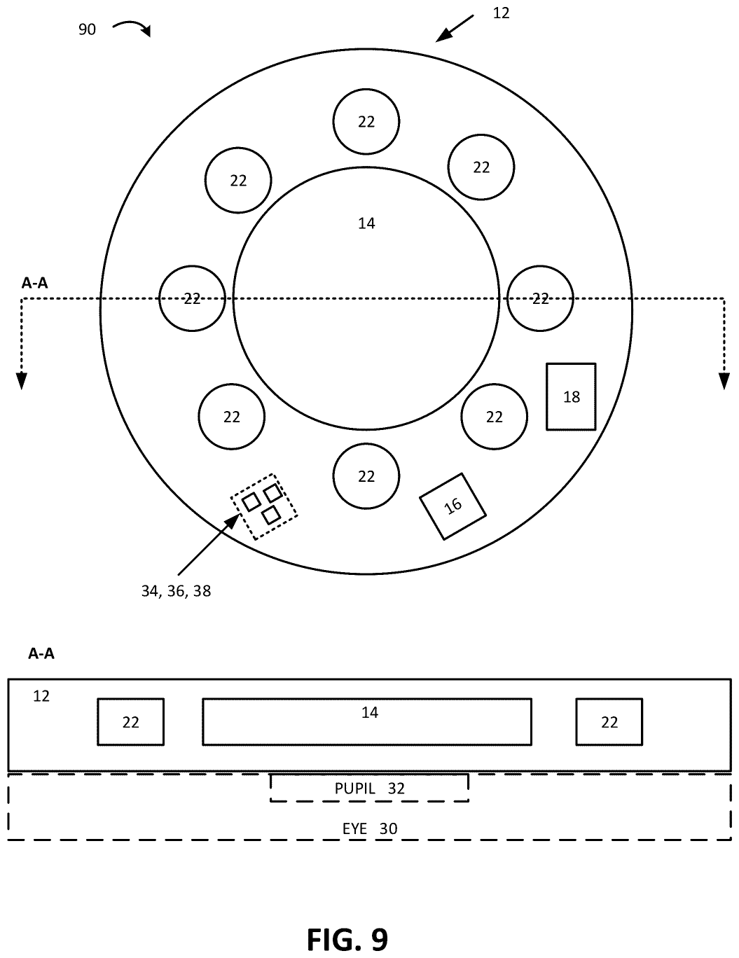

FIG. 9 shows one example embodiment of a system 90 where the ophthalmic device 12 is positioned on the surface of an eye 30. The ophthalmic device 12 can include at least a plurality of drug reservoirs 22 (that may be micro-sized to hold a dose of the therapeutic agent (not shown in FIG. 9)) embedded within the body of the ophthalmic device, but any number that is one or greater can be included based on the needs of a wearer (e.g., daily, weekly, or monthly contact lenses). The drug reservoirs 22 can be positioned within the ophthalmic device 12 so as to not occlude any portion of a user's eyesight when the ophthalmic device is in place on the eye. The therapeutic agent stored in the at least one drug reservoir 22 can be a muscarinic receptor antagonist for preventing or slowing the progression of myopia. For example, the therapeutic agent can be one or more of atropine, pirenzepine, tropicamide, or scopolamine. The electronically-mediated medium 14 is shown as a full circle embedded fully within the body of the ophthalmic device 12 in a central location that is in front of a lens of the eye 30 and entirely blocks the pupil 32 (even when the pupil 32 is dilated fully). In other examples, not shown the electronically-mediated medium 14 may be any shape or positioned such that at least a portion of the electronically-mediated medium can be in front of a lens of the eye 30 and reduce the effects of photophobia. When the electronically-mediated medium 14 is activated, then the light transmissions therethrough to the pupil 32 are reduced. The signal generator 16 can also be encapsulated within the body of the ophthalmic device 12 so as to not occlude a user's vision. The controller 18 is shown as embedded within the body of the ophthalmic device, but at least a part of the controller 18 could be external to the ophthalmic device 12. The ophthalmic device 12 can also include the ambient light sensor 34, a battery 36 configured to power the controller 18 (if controller 18 is embedded) and the signal generator 16, and a wireless transmitter 38 configured to transmit signals to and from an external handheld controller (could be controller 18 or another controller, e.g., a mobile device) where the external handheld controller can send instructions to the controller of the ophthalmic device. It should be understood that the external handheld controller can include a non-transitory memory and a processor.

### IV. Methods

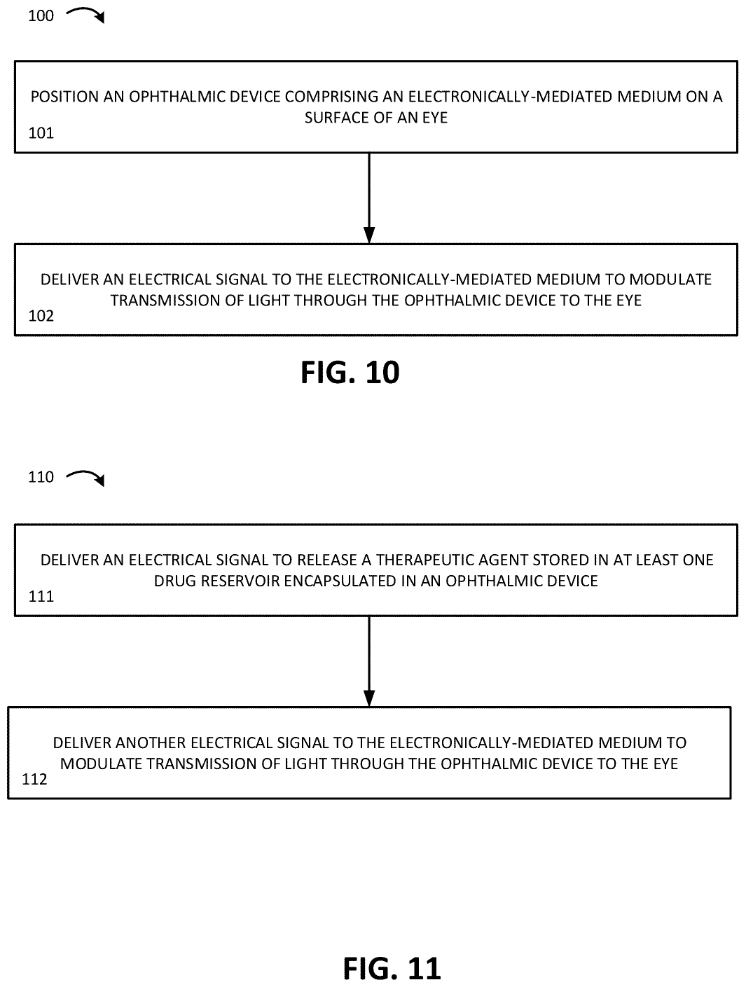

Another aspect of the present disclosure can include methods 100, 110, 120, and 130 (FIGS. 10-13) that can modulate an amount of light transmitted through an ophthalmic device (e.g., a smart contact lens). In some instances, the light can be modulated based on a delivery of an effective and timely dose of a therapeutic agent that can prevent or slow the development of myopia to prevent photophobia symptoms that may be caused be the therapeutic agent. The methods can be executed by the systems 10, 20, and 90 of FIGS. 1, 2, and 9, for example, using the control techniques of FIGS. 5-7.

The methods 100-130 are illustrated as process flow diagrams with flowchart illustrations that can be implemented by one or more components of the systems 10, 20, and 90. For purposes of simplicity, the methods 100-130 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 100-130.

FIG. 10 illustrates a method 100 for modulating light transmission through at least a portion of an ophthalmic device to an eye. At 101, the ophthalmic device can be positioned on a surface of an eye. The ophthalmic device can be a smart contact lens comprising an electronically-mediated medium within the body of the device, and other components in electrical communication with each other and the electronically-mediated medium such as a signal generator, a controller, a battery, a transmitter, an ambient light sensor, etc. The electronically-mediated medium can include a material that is at least one of a material that is one of electrochromic, liquid crystal, pH sensitive, or thermodynamic. A controller (embedded or external) can also be in communication with one or more of the components within the ophthalmic device. Upon positioning within the eye, the ophthalmic device can have a given light transmissibility. At 102, an electrical signal can be delivered, via the signal generator, to the electronically-mediated medium (e.g., to one or more conducting electrodes of the electronically-mediated medium) to modulate transmission of light through at least a portion of the ophthalmic device to the eye. The modulation can filter more light, so less light can reach the eye, if the ambient light is brighter than a given baseline light (e.g., normal indoor lighting) and can filter less light, so that more light (e.g., up to a normal atmospheric amount) of light can reach the eye, when the ambient light is darker than either the previous light or the baseline light. The electrical signal can be delivered as part of an automatic response to a change in the detected ambient light. Alternatively, the electrical signal can be delivered in response to a command from the user of the ophthalmic device (e.g., through a controller with a user interface, such as a mobile device, in communication with the signal generator). In this scenario the user can determine when and how much they desire to modulate the transmissibility of light through the one or more ophthalmic device the user is wearing. The electrical signal can be at least partially an AC signal with a magnitude and a frequency or a DC signal with a constant voltage or a variable voltage, both at a polarity

FIG. 11 illustrates a method 110 for delivering a therapeutic agent to an eye of a user and modulating an amount of light entering the eye through a smart ophthalmic device. At 111, an electrical signal can be delivered to release a therapeutic agent stored in at least one drug reservoir encapsulated in the ophthalmic device. The therapeutic agent can be delivered to the eye as a treatment to prevent and/or slow the progression of myopia. At least one parameter (e.g., magnitude, amplitude, frequency, polarity, wave shape, etc.) of an electrical signal can be defined, such as by a controller, so that when applied the electrical signal can release a therapeutic agent stored in at least one drug reservoir encapsulated in an ophthalmic device. The electrical signal can be generated, such as by a signal generator. The electrical signal can be delivered to a metal electrode of the at least one drug reservoir to electrodissolve the metal electrode and release the therapeutic agent. The metal electrode of the at least one drug reservoir can act as a removable cover for a well that stores the therapeutic agent in the drug reservoir. The released therapeutic agent can then diffuse, or move in another fashion, through the ophthalmic device and onto and/or into the eye wearing the ophthalmic device. At 112, another electrical signal can be delivered to the electronically-mediated medium to modulated transmission of light through the ophthalmic device to the eye.

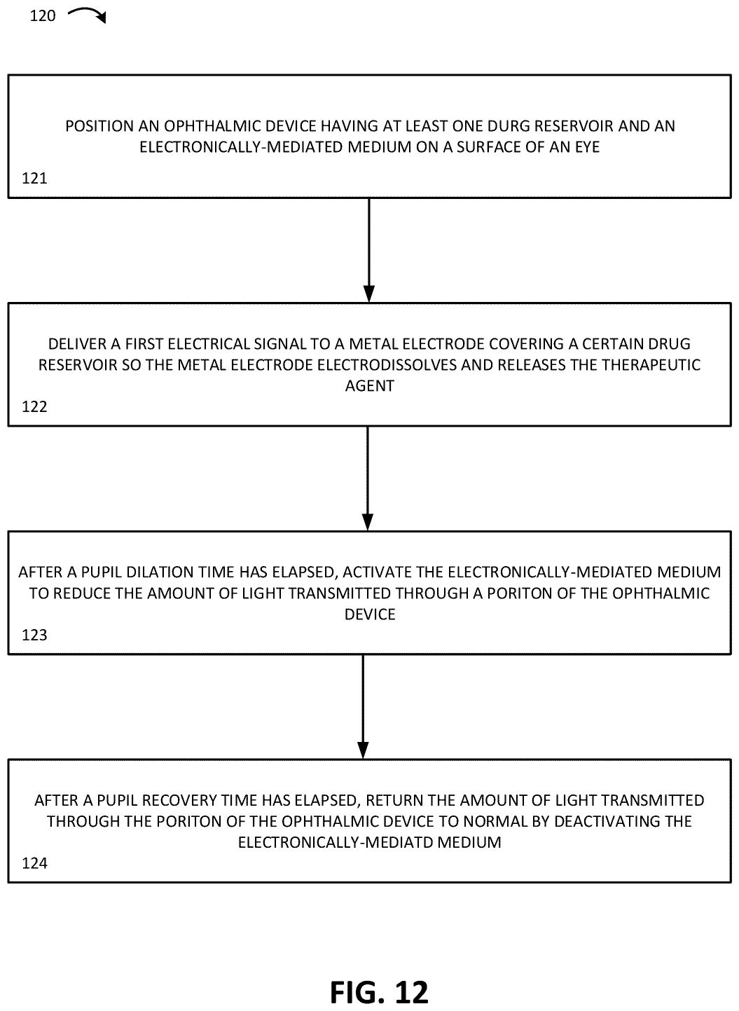

FIG. 12 illustrates a method 120 for providing a dose of a therapeutic agent to slow the progress of and/or prevent myopia and preventing the effects of the photophobic side effects of the therapeutic agent using a time-based control loop. At 121, an ophthalmic device, having at least one encapsulated drug reservoir configured to store a therapeutic agent covered by a metal electrode, can be positioned on a surface of an eye. The ophthalmic device can include an electronically-mediated medium. At 122, a first electrical signal can be delivered, via a signal generator, to the metal electrode covering a certain drug reservoir of the at least one encapsulated drug reservoir so the metal electrode undergoes electrodissolution to release the therapeutic agent. The therapeutic agent can cause dilation of a pupil of the eye it is delivered to. At 123, after a pupil dilation time has elapsed, the electronically-mediated medium can be activated to reduce the amount of light transmitted through a portion of the ophthalmic device by filtering the light. The electronically-mediated medium can activate in response to receiving a first portion of a second electrical signal from the signal generator. The first portion of the second electrical signal can be an AC signal with a magnitude and a frequency or a DC signal with a constant voltage or a variable voltage, both at a polarity. At 124, after a pupil recovery time has elapsed, the amount of light transmitted through the portion of the ophthalmic device can be returned to normal by deactivating the electronically-mediated medium. The electronically-mediated medium can deactivate in response to receiving a second portion of the second electrical signal. The second portion of the second electrical signal can have a zero voltage or a voltage of an opposite polarity depending on the first portion of the second electrical signal.

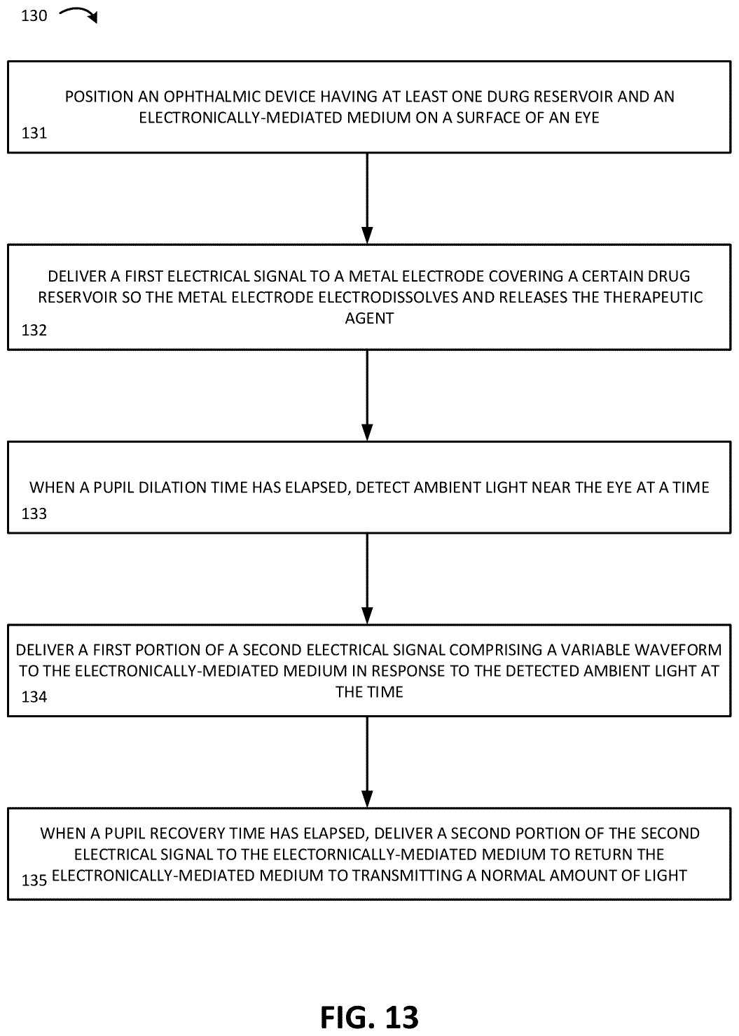

FIG. 13 illustrates a method 130 for providing a dose of a therapeutic agent to slow the progress of and/or prevent myopia and preventing the effects of the photophobic side effects of the therapeutic agent using an environment-based control loop. At 131, an ophthalmic device, having at least one encapsulated drug reservoir configured to store a therapeutic agent covered by a metal electrode, can be positioned on a surface of an eye. The ophthalmic device can include an electronically-mediated medium. At 132, a first electrical signal can be delivered, via a signal generator, to the metal electrode covering a certain drug reservoir of the at least one encapsulated drug reservoir so the metal electrode undergoes electrodissolution to release the therapeutic agent. The therapeutic agent can cause dilation of a pupil of the eye it is delivered to. At 133, when a pupil dilation time has elapsed, ambient light near the eye can be detected at a given time. Then, at 134, a first portion of a second electrical signal, comprising a variable waveform, can be delivered, via the signal generator, to the electronically-mediated medium in response to the detected ambient light at the time. The electronically-mediated medium can undergo a change to modulate an amount of light transmitted through at least a portion of the ophthalmic device. The electronically-mediated medium can activate in response to receiving a first portion of a second electrical signal from the signal generator. At 135, after a pupil recovery time has elapsed, a second portion of the second electrical signal can be delivered, via the signal generator, to the electronically-mediated medium to return the electronically-mediated medium to transmitting a normal amount of the light through the portion of the ophthalmic device. The second portion of the second electrical signal can have a zero voltage or a voltage of an opposite polarity depending on the first portion of the second electrical signal.

Before delivering the first portion of the second electrical signal the variable waveform can be configured, via the controller, based on if it is above or below a threshold or in a sliding scale based on the illuminance. For example, method 130 can further include steps to determine whether the detected ambient light at the time is above or below a threshold, and if the detected ambient light is above the threshold, then configure the variable waveform of the first portion of the second electrical signal to reduce the amount of light transmitted through the portion of the ophthalmic device by the electronically-mediated medium. If the detected ambient light is below the threshold, then configure the variable waveform of the first portion of the second electrical signal to return the amount of light transmitted through the portion of the ophthalmic device by the electronically-mediated medium to normal. In another example, method 130 can further include steps to determine the illuminance of the detected ambient light at the time and configure the variable waveform of the first portion of the second electrical signal to modulate the amount of light in inverse proportion to the illuminance of the ambient light detected at the time, wherein the variable waveform is a step function or a ramp function.

From the above description, those skilled in the art will perceive improvements, changes, and modifications. Such improvements, changes and modifications are within the skill of one in the art and are intended to be covered by the appended claims. All patents, patent applications, and publications cited herein are incorporated by reference in their entirety.

What is claimed is:

1. A system comprising:
an ophthalmic device, positionable on a surface of an eye, comprising an electronically-mediated medium configured to modulate an amount of light transmitted through a portion of the ophthalmic device in response to a first electrical signal; and
a signal generator encapsulated within the ophthalmic device, the signal generator configured to generate the first electrical signal,
wherein the signal generator is in communication with a controller, comprising a processor, configured to define parameters of the first electrical signal.

2. The system of claim 1, further comprising at least one drug reservoir encapsulated within the ophthalmic device, the at least one drug reservoir configured to store a therapeutic agent and to release the therapeutic agent in response to a second electrical signal,
wherein the signal generator is further configured to generate the second electrical signal to release the therapeutic agent, and
wherein the controller is further configured to define parameters of the second electrical signal.

3. The system of claim 2, wherein the therapeutic agent is a muscarinic receptor antagonist.

4. The system of claim 3, wherein the therapeutic agent is one of atropine, pirenzepine, tropicamide, or scopolamine.

5. The system of claim 2, wherein each drug reservoir comprises:
a well having an interior configured to hold the therapeutic agent in a non-liquid form;
a metal electrode configured to cover an opening of the well and to electrodissolve in response to receiving, from the signal generator, the second electrical signal.

6. The system of claim 5, wherein the processor of the controller executes instructions to:
apply the second electrical signal, generated by the signal generator, to the at least one drug reservoir to activate electrodissolution of the metal electrode so as to release of the therapeutic agent;
determine whether a pupil dilation time for the therapeutic agent has elapsed since release of the therapeutic agent started, wherein the therapeutic agent causes dilation of a pupil;
when the pupil dilation time has elapsed, apply a first portion of the first electrical signal to the electronically-mediated medium to modulate the amount of the light transmitted through the portion of the ophthalmic device;
determine whether a pupil dilation recovery time has elapsed; and
apply a second portion of the first electrical signal to the electronically-mediated medium to return the electronically-mediated medium to transmitting a normal amount of the light through the portion of the ophthalmic device.

7. The system of claim 6, wherein the first portion of the first electrical signal is applied for a time to the electronically mediated medium to reduce the amount of light transmitted through the portion of the ophthalmic device.

8. The system of claim 6, further comprising an ambient light sensor.

9. The system of claim 8, wherein the controller executes the instructions to:
after the pupil dilation time has elapsed, detect ambient light via the ambient light sensor at a time; and
modulate the amount of light transmitted through the portion of the ophthalmic device in response to the detected light.

10. The system of claim 9, wherein the amount of light transmitted through the portion of the ophthalmic device is modulated in response to the detected ambient light further comprises:
determine whether the detected ambient light is above a threshold value at the time,
if the detected ambient light is above the threshold value, then configure the first portion of the first electrical signal to reduce the amount of light transmitted through the portion of the ophthalmic device by the electronically-mediated medium; and
if the detected ambient light is below the threshold value, then configure the first portion of the first electrical signal to return the amount of light transmitted through the portion of the ophthalmic device by the electronically-mediated medium to normal.

11. The system of claim 9, wherein modulating the amount of light transmitted through the portion of the ophthalmic device in response to the detected ambient light further comprises:
determine the illuminance of the detected ambient light at the time; and
configure the first portion of the first electrical signal to reduce the amount of light in inverse proportion to the illuminance of the ambient light detected at the time.

12. The system of claim 11, wherein the first portion of the first electrical signal is a step function.

13. The system of claim 11, wherein the first portion of the first electrical signal is a ramp function.

14. The system of claim 1, wherein the electronically-mediated medium comprises a material that is one of electrochromic, liquid crystal, pH sensitive, or thermodynamic.

15. The system of claim 1, further comprising:
a battery configured to power the controller and the signal generator; and
a wireless transmitter configured to transmit to an external handheld controller, wherein the external handheld controller can send instructions to the controller of the ophthalmic device.

16. The system of claim 1, wherein when the ophthalmic device is positioned on a surface of the eye at least a portion of the electronically-mediated medium is configured to be in front of a lens of the eye.

17. A method comprising:
positioning an ophthalmic device, having at least one encapsulated drug reservoir configured to store a therapeutic agent covered by a metal electrode, on a surface of an eye, wherein the ophthalmic device comprises an electronically-mediated medium;
delivering a first electrical signal to the metal electrode covering a certain drug reservoir of the at least one encapsulated drug reservoir so the metal electrode undergoes electrodissolution to release the therapeutic agent, wherein the therapeutic agent is configured to cause dilation of a pupil;
after a pupil dilation time has elapsed, activating the electronically-mediated medium to reduce the amount of light transmitted through a portion of the ophthalmic device by filtering the light, wherein the electronically-mediated medium activates in response to receiving a first portion of a second electrical signal; and
after a pupil recovery time has elapsed, returning the amount of light transmitted through the portion of the ophthalmic device to normal by deactivating the electronically-mediated medium, wherein the electronically-mediated medium deactivates in response to receiving a second portion of the second electrical signal having a zero voltage or a voltage of an opposite polarity.

18. A method comprising:
positioning an ophthalmic device, having at least one encapsulated drug reservoir configured to store a therapeutic agent covered by a metal electrode, on a surface of an eye, wherein the ophthalmic device comprises an electronically-mediated medium;
delivering a first electrical signal to the metal electrode covering a certain drug reservoir so the metal electrode undergoes electrodissolution to release the therapeutic agent, wherein the therapeutic agent is configured to cause dilation of a pupil;
when a pupil dilation time has elapsed:
detecting ambient light near the eye at a time, and
delivering a first portion of a second electrical signal, comprising a variable waveform, to the electronically-mediated medium in response to the detected ambient light at the time, wherein the electronically-mediated medium undergoes a change to modulate an amount of light transmitted through at least a portion of the ophthalmic device; and
when a pupil recovery time has elapsed, delivering a second portion of the second electrical signal to return the electronically-mediated medium to transmitting a normal amount of the light through the portion of the ophthalmic device.

19. The method of claim 18, further comprising:
determining whether the detected ambient light at the time is above or below a threshold;
if the detected ambient light is above the threshold, then configuring the variable waveform of the first portion of the second electrical signal to reduce the amount of light transmitted through the portion of the ophthalmic device by the electronically-mediated medium; and
if the detected ambient light is below the threshold, then configuring the variable waveform of the first portion of the second electrical signal to return the amount of light transmitted through the portion of the ophthalmic device by the electronically-mediated medium to normal.

20. The method of claim 18, further comprising:
determining the illuminance of the detected ambient light at the time; and
configuring the variable waveform of the first portion of the second electrical signal to modulate the amount of light in inverse proportion to the illuminance of the ambient light detected at the time, wherein the variable waveform is a step function or a ramp function.
